(19) United States
(12) Patent Application Publication
(10) Pub. No.: US 2002/0173053 A1
(43) Pub. Date: Nov. 21, 2002

(54) Multiple simultaneous antigen detection by immunohistochemistry

(76) Inventors: Bassam Damaj, San Diego, CA (US); Ali-Samer Al-Assaad, San Diego, CA (US)

Correspondence Address: TRASK BRITT, P.O. BOX 2550, SALT LAKE CITY, UT, 84110, US

(21) Appl. No.: 09/947,726

(22) Filed: Sep. 6, 2001

Related U.S. Application Data
(60) Provisional application No. 60/286,924, filed on Apr. 27, 2001.

(51) Int. Cl. G01N 33/532

(52) U.S. Cl. 436/544

(57) ABSTRACT

A method of simultaneously identifying two different markers in a sample by contacting a sample suspected of containing the two different markers with an antibody solution having a first antibody or fragment thereof against a first marker and a second antibody or fragment thereof against a second marker. The first antibody is conjugated to a first enzyme and the second antibody is conjugated to a second enzyme. The first enzyme and the second enzyme are different. The sample is incubated with the antibody solution for a sufficient time to allow the first marker to bind with the first conjugated antibody and the second marker to bind with the second conjugated antibody. The sample is assayed for a change in enzymatic activity and the presence of the first marker and/or the second marker is determined. The present invention also includes a kit for the simultaneous detection of multiple target molecules.

### CROSS REFERENCE TO RELATED APPLICATIONS

### Related applications

Under the provisions of 35 U.S.C. §119(e), priority is claimed from Provisional Patent Application Serial No. 60/286,924 filed Apr. 27, 2001.

### TECHNICAL FIELD

The present invention relates to the field of immunohistochemistry and, more particularly, to the formation of antigen-antibody complexes for simultaneously identifying multiple antigens or markers in a pathological, or normal, specimen.

### BACKGROUND

Immunofluorescence is a commonly used technique for identifying antigens in a biological sample. Recently, detection and/or quantification of analytes, or target molecules, in a pathological specimen has become increasingly dependent on immunohistochemistry (“IHC”) techniques. IHC detects target molecules through antigen-antibody complexes in a pathological specimen using enzyme-linked antigens or antibodies. The presence of the target molecule is detected via an enzyme immunoassay.

A multitude of benefits are realized with IHC versus traditional immunofluorescence. For example, unlike immunofluorescence, IHC can be used with commonly used formalin-fixed paraffin-embedded tissue specimens. Pathological specimens, including histological tissue sections and/or other biological preparations such as tissue culture cells and PAP smears, are commonly used in diagnostic pathology and can be easily screened via IHC. Further, IHC staining is permanent and preserves cell morphology. A comparison of the cell morphology and antigen proliferation on two different slides can be useful in monitoring the progression of a disease.

The results of immunofluorescence are commonly read by flow cytometry which is relatively expensive to set up and perform. By way of contrast, IHC allows direct visualization. In fact, many non-specific diagnoses using flow cytometry may be distinguished through IHC.

Briefly, in IHC, a primary antibody that recognizes the target molecule of interest is introduced to a pathological specimen. The primary antibody binds to the target molecule in or on the pathological specimen. After incubation, a wash is performed to remove unbound antibody. Then, a secondary antibody, directed against the primary antibody and labeled with an enzyme, is incubated with the pathological specimen. During incubation, the secondary antibody will bind to the primary antibody. In another method, the primary antibody, which recognizes the target molecule, is labeled with the enzyme and no secondary antibody is used. Alternatively, the labeled antibody can be labeled with biotin rather than an enzyme. Then, in an additional step, enzyme-labeled avidin or streptavidin is introduced to the sample and allowed to bind to the biotionylated antibody.

Once a labeled antibody has been attached, either directly or indirectly, to the specimen, a substrate, specific for the enzyme, is added to the specimen. When the substrate is added, the enzyme label converts the substrate causing a color change that can be seen with light microscopy. The presence of a color change indicates the presence of the target molecule and allows an observer to determine, assess, and diagnose the disease level and severity.

IHC has been used in a wide variety of immunodiagnostic applications, such as serodiagnostics, to detect antigens from a wide range of specific viruses, bacteria, fungi and parasites, and to measure the presence of antibodies against these various microorganisms. Similarly, these techniques can monitor factors involved in non-infectious diseases such as hormone levels, hematological factors, serum tumor markers, drug levels and antibody levels. IHC can be used to classify and diagnose poorly differentiated malignant tumors. Further, IHC permits the identification of the primary site or origin of metastatic tumors as tumors usually contain markers which identify the site of origin.

Current immunohistochemistry techniques are limited as they are only capable of detecting one target molecule at a time. However, it is common for several antigenic substances or markers to be associated with a pathological or physiological disorder. Thus, in order to accurately diagnose or monitor a condition, a different specimen would have to be prepared for each target molecule to be detected. Such a process can be difficult especially in the case of cancer diagnosis and treatment as the tissue sample often contains only a very small amount of tissue that was obtained, for example, from a needle tissue biopsy. Further, the multitude of tests and specimens increase the cost, time, and the possibility of analytical error. Similarly, researchers are often interested in more than one target molecule within a particular specimen. Thus, it would be advantageous to have an immunohistochemical assay for the simultaneous detection of multiple antigens within a single pathological specimen.

### DISCLOSURE OF THE INVENTION

The invention includes a new immunohistochemical assay used in enzyme immunochemical techniques for simultaneously and rapidly detecting more than one marker or antigen on the same histological tissue section and/or any other biological preparations. The positive staining of two markers simultaneously will help the pathologist/scientist to evaluate and diagnose the disease condition very rapidly, and allows visualization and comparison of the intensity of the markers simultaneously on the same tissue section.

One embodiment of the invention includes a method of simultaneously identifying two different markers in a sample by contacting a sample suspected of containing the two different markers with an antibody solution having a first antibody against a first marker and a second antibody against a second marker. The first antibody is conjugated to a first enzyme and the second antibody is conjugated to a second enzyme. The first enzyme and the second enzyme are different. The sample is incubated with the antibody solution for a sufficient time to allow the first marker to bind with the first conjugated antibody and the second marker to bind with the second conjugated antibody. The sample is assayed for a change in enzymatic activity and the presence of the first marker and/or the second marker is determined.

Another embodiment of the invention includes a method of simultaneously determining the presence of an antigen and a disease marker in a tissue sample suspected of containing the antigen and the disease marker by incubating an enzyme-conjugated antibody mix with the tissue sample, the enzyme-conjugated antibody mix including a first antibody against the antigen, the first antibody conjugated to a first enzyme, and a second antibody against the disease marker, the second antibody conjugated to a second enzyme. The tissue sample is washed and a first substrate specific for the first enzyme and then a second substrate specific for the second enzyme are sequentially added to the tissue sample. The presence of the antigen and the disease marker in the tissue sample is determined.

Another embodiment of the invention includes a kit for use in the simultaneous identification of a first target molecule and a second target molecule in a pathological tissue sample. The kit includes an antibody solution including a first antibody which immunologically recognizes the first target molecule, the first antibody conjugated to a first means of detection, and a second antibody which immunologically recognizes the second target molecule, the second antibody conjugated to a second means of detection. The kit also includes a first reagent reactive with the first means of detection to produce a detectable reaction product and a second reagent reactive with the second means of detection to produce a detectable reaction product. The invention thus includes methods of making the kit according to the invention.

### BRIEF DESCRIPTION OF THE FIGURES

The file of this patent contains at least one drawing executed in color. Copies of this patent with color drawing(s) will be provided by the Patent and Trademark Office upon request and payment of the necessary fee.

FIG. 1 illustrates positive staining for keratin in basal cells as shown by the heavy black lines.

FIG. 2 depicts positive staining for PSA in epithelial cells as shown by golden-orange staining and the black arrows.

FIG. 3 depicts simultaneous staining of the epithelial cells (PSA) and basal cells (keratin).

FIG. 4 is a negative control illustrating a section where no staining is seen.

### DETAILED DESCRIPTION OF THE INVENTION

The present invention allows simultaneous detection of multiple target molecules on the same pathological specimen. As used herein, the term “target molecule” refers to antibodies or antigens detectable by the present invention. Target molecules include, but are not limited to, antigens, including antigens derived from microorganisms and other pathogens, antibodies produced in response to those antigens, tumor markers, proteins, receptors, DNA, RNA and any artificial nucleic acid molecules, fragments or probes, or other oligonucleotides, and self-antibodies generated in autoimmune disease.

Pathological specimens, include but are not limited to, a histological tissue section and/or other biological preparations such as tissue culture cells and PAP smears.

Current IHC methods are only capable of detecting one target molecule at a time. Several antigenic substances or markers are often associated with a pathological or physiological disorders and can be early indicators of disease. Hence, the accurate diagnosis of infection or disease may require several different specimens and several different screening processes.

For example, chlamydia and gonorrhea infections are often coincident in women. It would be advantageous to be able to test and detect both infections with the same sample. Similarly, the detection, diagnosis and monitoring of prostate cancer can be assisted with the screening of additional markers. For example, prostate specific antigen (“PSA”) is normally produced by the body. Thus, a mildly increased PSA level is insufficient to support a diagnosis of cancer. The absence of a basal cell layer is a well accepted criterion for diagnosis of prostate carcinoma. However, it can be difficult to identify this cell layer on standard histological examinations. IHC staining antibody to a particular keratin can help to identify a fragmented basal cell layer which assists in the diagnosis of carcinoma. The present invention permits the simultaneous detection of PSA and high molecular weight keratin on the same histological sample.

Although the invention is described herein using detection antibodies, those skilled in the art will understand that it is also applicable to any antagonist, which recognize the target molecule to be detected. Any antagonist including antigens, primers, nucleic acids, or fragments thereof, that recognize specific proteins, markers, receptors or antibodies, or fragments thereof, to be detected may be used. For example, if a particular nucleic acid sequence is the target molecule, an artificial or naturally occurring sequence having affinity for the target nucleic acid sequence or detection antibody may be used. The detecting sequence need not necessarily be complementary to the target sequence. The detecting sequence should have sufficient affinity for the target sequence so that the two sequences remain bound during the detection process.

When using antigens to detect target molecules, the antigens are allowed to react to a tissue section. Then, a first enzyme conjugated to a secondary antibody specific for a first antigen and a second enzyme conjugated to a secondary antibody specific for a second antigen are added to the tissue section. After sufficient incubation, a enzyme substrate specific for the first enzyme is added. After sufficient incubation, a second enzyme substrate specific for the second enzyme is added. The changes in colors that are visualized under light microscopy indicate the presence of the target molecules.

General Description of the Multiple Simultaneous Antigen Detection by Immunohistochemistry

Conjugation of antibodies.

Detection antibodies to anti-human marker₁ antibody (“antibody 1”) and anti-human marker₂ antibody (“antibody 2”), are conjugated to different enzymes. For example, antibody 1 was conjugated to horseradish peroxidase (“HRPO”) and antibody 2 was conjugated to alkaline phosphatase (“AKP”). Conjugation of an antibody to an enzyme was accomplished as follows. 5 mg/ml of an antibody solution was dialyzed in 0.1 M phosphate buffer at pH 6.8 (PBS) overnight at 4° C. 0.5 mg of the dialyzed antibody solution was added to 1.5 mg of the enzyme in 10 ml of 10 mM PBS. 80 μl 25% glutaraldehyde was added and the solution was mixed gently. The solution was allowed to stand at room temperature for 2 hours. The reaction was stopped by adding an equivalent volume (10 ml) of PBSLE (10 mM PBS containing 100 mM lysine and 100 mM ethanolamine). The solution was de-salted with a Sephadex G25 column in PBSN (10 mM PBS with 0.05M NaN₃). 20 ml of the enzyme-antibody conjugate was mixed with 40 ml of blocking buffer (0.17 M borate buffer containing 2.5 mM MgCl₂, 0.05% Tween 20, 1 mM EDTA, 0.25% BSA and 0.05% NaN₃). 60 mL of the enzyme-antibody conjugate was filtered through a low-protein binding filter, Millex HV 0.45 μm, for sterilization and the solution was stored at 4° C.

Tissue sections.

Slides were obtained from UCSD Medical Center Pathology Lab, where biopsies were covered with OCT, frozen and cut using a cryostat. Sections were placed on positively charged slides for further analysis.

Immunohistochemistry.

Step 1.

Paraffin and OCT were removed first by very briefly placing the slides on a hot plate. Slides were then placed into xylene for 3 minutes and followed in 100% ethanol for 3 minutes. The slides were then placed in water for 1 minute to remove OCT and then washed in TBS for 5 minutes.

Step 2.

Sections were then permeabilized either by being placed in trypsin for 1.5 hours at 37° C. or by being placed in 00.01-0.05% n-Octyl β-_{D}-Glucopyranoside in trypsin for 2 hours at 37° C. Slides were then washed twice in TBS for 5 minutes each time.

Step 3.

200 μl of an enzyme-conjugated antibody mix was added to slide. The enzyme-conjugated antibody mix contains conjugated antibodies, for example, anti-PSA antibody conjugated to HRPO and anti-keratin antibody conjugated to AKP. The slides were incubated overnight at 4° C. Slides were then washed twice in TBS for 5 minutes each time.

Step 4.

100 μl of a HRPO specific substrate, such as 3, 3′-diaminobenzidine (“DAB”), was added to the specimen for 10 minutes or longer at room temperature. The slide was rinsed by dipping it in TBS. 100 μl of AKP specific substrate, such as BCIP/NBT, was added for 20 minutes or longer at room temperature. Slides were then washed twice in TBS for 5 minutes each time.

Step 5.

The slides were counterstained by covering the sample with methyl green crystal violet free dye for 2 minutes. The reaction was stopped by washing the slide in distilled water. The slide was mounted using a mounting medium as known in the art.

The invention may be further understood by reference to the non-limiting examples set forth below.

### EXAMPLES

Simultaneous Antigen Detection for Prostate Specific Antigen and Keratin by Immunohistochemistry

Procedure.

The assay of the present invention involves the simultaneous detection of two antigens on the same pathological sample, on the same slide and the same time. The assay procedure was as described in the Immunohistochemistry section above.

Slides containing a histological sample were preheated to melt the paraffin or OCT and then incubated in xylene and 100% ethanol respectively to dehydrate the slide. To remove any OCT residue, the slides were then washed with water. A washing step was performed prior to tissue permeabilization. The tissues were permeabilized using either typsin for 1.5 hours at 37° C. or 0.01-0.05% n-Octyl β-_{D}-Glucopyranoside in trypsin for 2 hours at 37° C. followed by a washing step.

The slides were incubated overnight at 4° C. with 200 μl of each enzyme-conjugated antibody mix. The enzyme-conjugated antibody mix contains conjugated antibodies, anti-PSA antibody conjugated to HRPO and anti-keratin antibody conjugated to AKP. The overnight incubation was followed by another washing step.

Specific substrates, DAB and BCIP/NBT, for HRPO and AKP, respectively, were added sequentially for about 10-15 minutes. A quick wash step occurs before the second substrate was added. The sequential addition of the substrates was important for the development of color that will be visualized under light microscopy as the buffer of one substrate can kill the enzyme of another substrate.

The slides were then counter-stained using an appropriate counter-stain and mounted for storage and future analysis.

Results

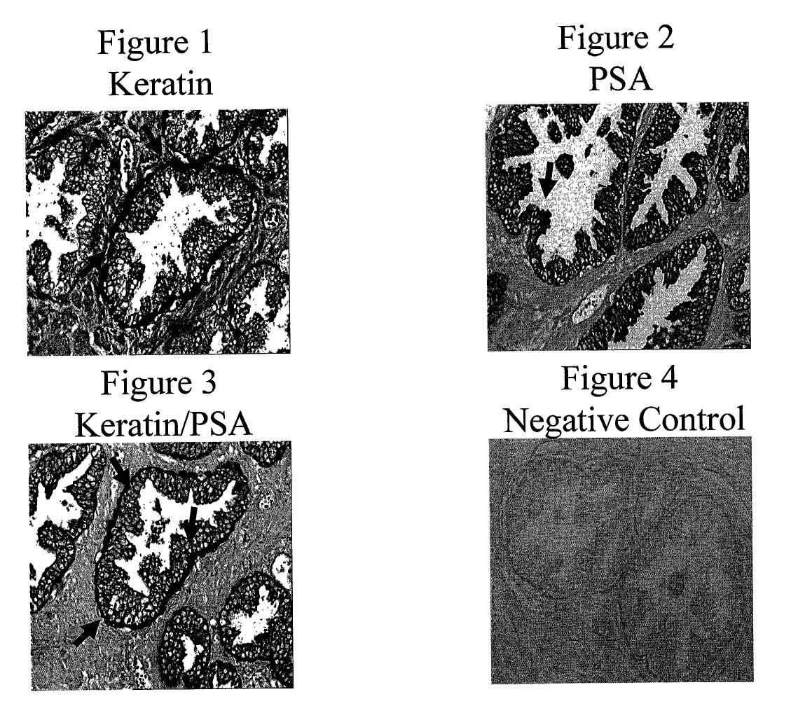

The results of the immunostaining are shown in FIG. 3. A negative control is shown in FIG. 4. The presence of PSA in epithelial cells is illustrated by a golden-orange staining and highlighted by the black arrows. (FIGS. 2 and 3). The presence of keratin in basal cells is indicated by black lines as illustrated in FIGS. 1 and 3. The present invention permits the use of several different markers or detection antibodies without adversely affecting the assay performance when compared to the measurement of one marker per slide as seen in FIGS. 1-3. Thus, visualization and detection of the target molecules is not impaired.

Although the foregoing description contains many specifics, these should not be construed as limiting the scope of the present invention, but merely as providing illustrations of some exemplary embodiments. Similarly, other embodiments of the invention may be devised which do not depart from the spirit or scope of the present invention. Features from different embodiments may be employed in combination. The scope of the invention is, therefore, indicated and limited only by the appended claims and their legal equivalents, rather than by the foregoing description. All additions, deletions, and modifications to the invention, as disclosed herein, which fall within the meaning and scope of the claims are to be embraced thereby.

What is claimed is:

1. A method of simultaneously identifying at least two different markers in a sample, said method comprising:
contacting a sample suspected of containing the at least two different markers with an antibody solution having a first antibody or fragment thereof against a first marker of said at least two different markers, said first antibody or fragment thereof conjugated to a first enzyme, and a second antibody or fragment thereof against a second marker of said at least two different markers, said second antibody or fragment thereof conjugated to a second enzyme, wherein said first enzyme and said second enzyme are different;
incubating said sample with said antibody solution for a sufficient time to allow the first marker to bind with the first conjugated antibody and the second marker to bind with the second conjugated antibody;
assaying the sample for a change in enzymatic activity; and
determining the presence of said first marker and said second marker.

2. The method according to claim 1, wherein said assaying comprises sequentially adding to said sample a first substrate specific for said first enzyme and then a second substrate specific for said second enzyme.

3. The method according to claim 2, wherein said antibody solution further comprises a third antibody or fragment thereof against a third marker of said at least two different markers, said third antibody or fragment thereof conjugated to a third enzyme.

4. The method according to claim 3, further comprising adding a third substrate specific for said third enzyme.

5. The method according to claim 2, wherein said first enzyme is alkaline phosphatase, said second enzyme is horseradish peroxidase, said first substrate is BCIP/NBT and said second substrate is DAB.

6. The method according to claim 1, wherein said first enzyme is selected from the group consisting of alkaline phosphatase, horseradish peroxidase, beta-galactosidase, beta-glucuronidase, luciferase, and urease.

7. The method according to claim 1, wherein the second enzyme is selected from the group consisting of alkaline phosphatase, horseradish peroxidase, beta-galactosidase, beta-glucuronidase, luciferase, and urease.

8. The method according to claim 1, wherein said change in enzymatic activity comprises a detectable color change.

9. The method according to claim 1, wherein the sample is a formalin-fixed paraffin-embedded tissue section.

10. The method according to claim 1, wherein said first marker is prostate specific antigen and said second marker is keratin.

11. A method of simultaneously determining the presence of an antigen and a disease marker in a tissue sample suspected of containing the antigen and the disease marker, said method comprising:
incubating an enzyme-conjugated antibody mix with said tissue sample, said enzyme-conjugated antibody mix including a first antibody against said antigen, said first antibody conjugated to a first enzyme, and a second antibody against said disease marker, said second antibody conjugated to a second enzyme;
washing said tissue sample;
adding a first substrate specific for said first enzyme and sequentially adding a second substrate specific for said second enzyme; and
determining the presence of said antigen and said disease marker in said tissue sample.

12. The method according to claim 11, wherein said determining comprises detecting enzymatic activity in said tissue sample.

13. The method according to claim 12, wherein said change in enzymatic activity comprises a detectable color change.

14. The method according to claim 11, wherein said first enzyme and said second enzyme are different and are selected from the group consisting of alkaline phosphatase, horseradish peroxidase, beta-galactosidase, beta-glucuronidase, luciferase, and urease.

15. The method according to claim 11, wherein said antigen is prostate specific antigen and said disease marker is keratin.

16. A kit for the simultaneous identification of a first target molecule and a second target molecule in a pathological tissue sample, said kit comprising:
an antibody solution including a first antibody or fragment thereof which immunologically recognizes said first target molecule, said first antibody or fragment thereof conjugated to a first means of detection, and a second antibody or fragment thereof which immunologically recognizes said second target molecule, said second antibody or fragment thereof conjugated to a second means of detection;
a first reagent reactive with said first means of detection to produce a detectable reaction product; and
a second reagent reactive with said second means of detection to produce a detectable reaction product.

17. The kit of claim 16, wherein said first target molecule is prostate specific antigen and said second target molecule is keratin.

18. The kit of claim 16, wherein said first means of detection is a first enzyme and said second means of detection is a second enzyme.

19. The kit of claim 18, wherein said first enzyme and said second enzyme are different and selected from the group consisting of alkaline phosphatase, horseradish peroxidase, beta-galactosidase, beta-glucuronidase, luciferase, and urease.

20. The kit of claim 16, further comprising one or more positive or negative control pathological tissue samples.

21. A method of making a kit for use in simultaneously identifying at least two target molecules in a sample, said method comprising:
providing an antibody solution including a first antibody or fragment thereof which immunologically recognizes a first target molecule, said first antibody or fragment thereof conjugated to a first means of detection, and a second antibody or fragment thereof which immunologically recognizes a second target molecule, said second antibody or fragment thereof conjugated to a second means of detection;
providing a first reagent reactive with said first means of detection to produce a detectable reaction product; and
providing a second reagent reactive with said second means of detection to produce a detectable reaction product.

22. The method according to claim 21, wherein said providing an antibody solution including a first antibody or fragment thereof comprises providing said antibody solution wherein said first antibody or fragment thereof immunologically recognizes prostate specific antigen and wherein said second antibody or fragment thereof immunologically recognizes keratin.

23. The method according to claim 21, wherein said first means of detection is a first enzyme and said second means of detection is a second enzyme.

24. The method according to claim 23, wherein said first enzyme and said second enzyme are different and selected from the group consisting of alkaline phosphatase, horseradish peroxidase, beta-galactosidase, beta-glucuronidase, luciferase, and urease.

25. The method according to claim 21, further comprising providing one or more positive or negative control samples.

26. A method of using a kit to simultaneously diagnose the presence of a disease, said method comprising:
providing a kit capable of simultaneously identifying at least two markers for a disease, said kit comprising an antibody solution including a first antibody or fragment thereof which immunologically recognizes a first marker of said at least two markers, said first antibody or fragment thereof conjugated to a first enzyme, and a second antibody or fragment thereof which immunologically recognizes a second marker of said at least two markers, said second antibody or fragment thereof conjugated to a second enzyme;
introducing a sample suspected of including said at least two markers to said antibody solution; and
performing an assay to simultaneously detect the presence of said at least two markers for said disease.

27. The method according to claim 26 wherein said performing an assay comprises:
introducing a first reagent to said sample to produce a detectable reaction product, wherein said first reagent is reactive with said first enzyme; and
introducing a second reagent to said sample to produce a detectable reaction product, wherein said second reagent is reactive with said second enzyme.

28. The method according to claim 26, wherein said first marker is prostate specific antigen and said second marker is keratin.

29. The method according to claim 28, wherein said first enzyme and said second enzyme are different and selected from the group consisting of alkaline phosphatase, horseradish peroxidase, beta-galactosidase, beta-glucuronidase, luciferase, and urease.
